(19) United States
(12) Patent Application Publication
(10) Pub. No.: US 2003/0077301 A1
(43) Pub. Date: Apr. 24, 2003

(54) Topical pharmaceutical composition for the treatment of inflammatory dermatoses

(76) Inventors: Howard I. Maibach, San Francisco, CA (US); Eric C. Luo, Plano, TX (US); Tsung-Min Hsu, San Diego, CA (US)

Correspondence Address: REED & EBERLE LLP, 800 MENLO AVENUE, SUITE 210, MENLO PARK, CA, 94025, US

(21) Appl. No.: 10/177,250

(22) Filed: Jun. 21, 2002

Related U.S. Application Data
(63) Continuation-in-part of application No. 09/972,008, filed on Oct. 4, 2001.
Application No. 09/972,008 is a continuation-in-part of application No. 09/738,410, filed on Dec. 14, 2000.
Application No. 09/738,410 is a continuation-in-part of application No. 09/569,889, filed on May 11, 2000.
Application No. 09/569,889 is a continuation-in-part of application No. 09/465,098, filed on Dec. 16, 1999, now abandoned.
Application No. 09/569,889 is a continuation-in-part of application No. 09/738,395, filed on Dec. 14, 2000.
Application No. 09/738,395 is a continuation-in-part of application No. 09/607,892, filed on Jun. 30, 2000, now abandoned.

(51) Int. Cl. A61K 31/7048; A61K 31/65; A61K 31/63; A61K 31/60; A61K 31/573; A61K 31/203; A61K 33/00; A61K 33/04

(52) U.S. Cl. 424/400; 424/722; 424/703; 514/29; 514/152; 514/159; 514/155; 514/171; 514/559; 514/568

(57) ABSTRACT

Provided is a topical pharmaceutical composition for the treatment of inflammatory dermatoses, including acne vulgaris, together with methods for its use. The composition and methods involve the topical use of an active agent effective in the treatment of inflammatory dermatoses plus a permeation-enhancing base that, in one embodiment, gives the composition a pH of about 8.0 to about 13.0, preferably about 8.0 to 11.5, and most preferably about 8.5 to 10.5.

### CROSS-REFERENCE TO RELATED APPLICATIONS

This application is a continuation in part of U.S. Ser. No. 09/972,008 filed on Oct. 4, 2001, which is a continuation in part of U.S. Ser. No. 09/738,410 filed on Dec. 14, 2000, which is a continuation in part of U.S. Ser. No. 09/569,889 filed on May 11, 2000, which is a continuation in part of U.S. Ser. No. 09/465,098 filed on Dec. 16, 1999; and is a continuation in part of U.S. Ser. No. 09/738,395 filed on Dec. 14, 2000, which is a continuation in part of U.S. Ser. No. 09/607,892 filed Jun. 30, 2000, now abandoned.

### TECHNICAL FIELD

This invention relates generally to methods and pharmaceutical formulations for treating inflammatory dermatoses, particularly acne vulgaris. More specifically, this invention relates to compositions that comprise (a) an active agent useful in the treatment of inflammatory dermatoses when applied topically, and (b) an agent that enhances the permeability of skin or mucosal tissue to the active agent.

### BACKGROUND

Acne vulgaris is a condition that affects nearly all individuals between the ages of 12 and 17, although some people continue to suffer from the condition well into their thirties or beyond. Acne lesions are commonly located on the face, but the lesions can also be found on the neck, chest, back, shoulders, scalp, upper arms, and legs. It has been estimated that Americans spend well over one hundred million dollars for the treatment of acne each year.

Acne occurs in response to clogged hair follicles. Initially, sebaceous glands associated with a hair follicle fill the follicle with sebum, an oil-like substance. Dead skin cells lining the hair follicle slough off into the follicle. Normally, sebum, dead skin cells, and other substances are routinely eliminated from the follicle. When the sebum and dead skin cells form a plug in the follicle, however, a comedone develops. An open comedone occurs when the opening of the follicular canal dilates and the plug protrudes form the canal, turning a characteristic dark color upon exposure to the air. Open comedones are also referred to as “blackheads” because of this dark color. Closed comedones occur when the follicle is covered, e.g., with a layer of cells, such that the plug does not reach the external environment. Closed comedones are also referred to as “whiteheads,” due to their characteristic white color.

The production of sebum and dead skin cells lining the follicle increases dramatically during puberty in response to hormonal changes. It is this increased production of these products that causes adolescents to be the most likely individuals to suffer from acne.

Plugs that are not removed from open and closed comedones may swell further. Additional sebum and dead skin cells may accumulate in the clogged follicle. In addition, bacteria such as Propionibacterium acnes may multiply, secreting enzymes that hydrolyze sebum into free fatty acids. The enzymes and the free fatty acids trigger an inflammatory process. As a result, white blood cells such as neutrophils migrate to the follicle, causing an erythematous papule to develop. With continued sebum production and bacterial growth, the follicle ruptures, causing its contents to spill into the dermis. Continuation of this severe inflammation can cause a cyst.

Based on its etiology, acne may be classified into one of three categories: comedonal, inflammatory, and nodulocystic. Comedonal acne consists predominately of open or closed comedones with little or no accompanying inflammation. Eythromatous papules and pustules characterize inflammatory acne, though comedones may also be present. Nodulocystic acne is characterized by deep nodules and cysts; other inflammatory lesions and comedones are also usually present.

Current Treatments:

Several drugs are currently in use to treat acne. Systemic therapy includes oral contraceptives, erythromycin, tetracycline, doxycycline, minocycline, and isotretinoin. Systemic therapy, however, has many drawbacks. For example, resistance may be developed to antibiotics such as erythromycin, tetracycline, doxycycline, and minocycline (Nishijima et al. (2000), J. Dermatol 27(5):318-323). Oral contraceptives are inappropriate for male patients as well as for certain populations of female patients, e.g., female patients with a history of breast carcinoma or thromboembolic disorders. Due to its teratogenic activity, isotretinoin therapy requires that female patients not become pregnant during treatment. Furthermore, systemic administration causes systemic side effects, as relatively high levels of the drug must circulate throughout the entire body.

Topical therapy addresses some of the concerns associated with systemic therapy and represents a useful approach to treating individuals suffering from acne. Topical agents employed to treat acne include antibiotics, retinoids, benzoyl peroxide, sulfur, and corticosteroids; combinations of such agents are commonly used. A single antibiotic is rarely used alone, to avoid the development of bacterial resistance; combinations, e.g., with benzoyl peroxide or a retinoid, are common. Some antibiotics used in topical acne treatments are erythromycin, azelaic acid, clindamycin, tetracycline, and sodium sulfacetamide. Benzoyl peroxide, which has some antibiotic activity, is commonly used in topical anti-acne preparations as a single agent or in combination with other agents. For example, U.S. Pat. No. 5,740,884 describes administering an anti-acne composition containing benzoyl peroxide, and U.S. Pat. No. 5,753,637 describes topical administration of a combination of benzoyl peroxide, salicylic acid, and a vasoconstrictor to treat an individual suffering from acne. Salicylic acid, which is keratolytic and helps to unclog pores, is commonly used to treat mild acne, particularly in combination with other agents. Sulfur has long been used in the treatment of acne. It appears to have keratolytic activity, though the mechanisms of its anti-acne activity are not well understood. It is generally used in combination with other agents, such as sodium sulfacetamide, alcohol, salicylic acid, and resorcinol (which is sometimes used alone). Retinoids, which are compounds closely related to vitamin A, are particularly effective against acne, though they have some significant potential adverse effects. These adverse effects include redness, dryness, peeling, and itching of the skin, and the potential for birth defects. Though the risk of birth defects is low for topically applied retinoids, these compounds are prescribed with caution to women who are pregnant or likely to become pregnant. Commonly used topical retinoids (and closely related compounds) include tretinoin, adapalene, and tazarotene. Corticosteroids are sometimes used in the topical treatment of acne, generally in conjunction with other agents. The corticosteroid triamcinolone is occasionally injected into acne lesions, though such injections commonly produce a temporary darkening of the surrounding skin. U.S. Pat. No. 5,958,984 to Devillez describes topically applying a composition containing hydrogen peroxide for the treatment of acne.

While topical administration of any one of these compositions may work for some individuals some of time, additional formulations for treating acne and other inflammatory dermatoses are desired. It has now been discovered that certain basic compositions, when used in conjunction with pharmaceutical agents active against inflammatory dermatoses, successfully treat inflammatory dermatoses, including sebaceous gland disorders such as acne vulgaris, without the pain, irritation, and other adverse effects experienced with other treatments. The present invention thus addresses needs in the art by providing a novel treatment for acne and other inflammatory dermatoses that is effective, safe, not painful, and convenient.

Skin Permeation Enhancement:

The delivery of drugs topically to the skin provides many advantages. For the patient, it is comfortable, convenient, and noninvasive. The variable rates of absorption and metabolism encountered in oral treatment are avoided, and other inherent inconveniences (e.g., gastrointestinal irritation, the need for administration with food in some cases or without food in other cases) are eliminated. Of particular interest in the treatment of acne and other inflammatory dermatoses, topical drug delivery permits localized treatment, so that only the affected areas of skin need be exposed to the drug. Such localized treatment avoids the incurring of high systemic drug levels and the consequent toxicity or other adverse effects that could follow.

The topical delivery of drugs into the skin, however, is commonly challenging. Skin is a structurally complex, relatively thick membrane. Molecules moving from the environment into and through intact skin must first penetrate the stratum corneum and any material on its surface. The stratum corneum is a layer approximately 10-15 micrometers thick over most of the body that consists of dense, highly keratinized cells. The high degree of keratinization within these cells, as well as their dense packing, are believed to be the factors most responsible for creating, in most cases, a substantially impermeable barrier to drug penetration. With many drugs, the rate of penetration through the skin is extremely low without the use of some means to enhance the skin's permeability. As the stratum corneum of many inflammatory dermatoses is commonly thicker than that of normal skin, the penetration of topical drugs into the affected areas of skin is particularly difficult to achieve.

In order to increase the degree and rate at which a drug penetrates the skin, various approaches have been followed, each of which involves the use of either a chemical penetration enhancer or a physical penetration enhancer. Physical enhancements of skin permeation include, for example, electrophoretic techniques such as iontophoresis. The use of ultrasound (or “phonophoresis”) as a physical penetration enhancer has also been researched. Chemical penetration enhancers are more commonly used. These are compounds that are topically administered along with a drug (or, in some cases, prior to drug administration) in order to increase the permeability of the stratum corneum, and thereby provide for enhanced penetration of the drug through the skin. Ideally, such chemical penetration enhancers (or “permeation enhancers,” as the compounds are referred to herein) are compounds that are innocuous and serve merely to facilitate diffusion of the drug through the stratum corneum.

Various compounds for enhancing the permeability of skin are known in the art and are described in the pertinent texts and literature. Compounds that have been used to enhance skin permeability include: sulfoxides such as dimethylsulfoxide (DMSO) and decylmethylsulfoxide (C₁₀MSO); ethers such as diethylene glycol monoethyl ether (available commercially as Transcutol®) and diethylene glycol monomethyl ether; surfactants such as sodium laurate, sodium lauryl sulfate, cetyltrimethylammonium bromide, benzalkonium chloride, Poloxamer (231, 182, 184), Tween (20, 40, 60, 80), and lecithin (U.S. Pat. No. 4,783,450); the 1-substituted azacycloheptan-2-ones, particularly 1-n-dodecylcyclazacycloheptan-2-one (available under the trademark Azone® from Nelson Research & Development Co., Irvine, Calif.; see U.S. Pat. Nos. 3,989,816, 4,316,893, 4,405,616, and 4,557,934); alcohols such as ethanol, propanol, octanol, benzyl alcohol, and the like; fatty acids such as lauric acid, oleic acid and valeric acid; fatty acid esters such as isopropyl myristate, isopropyl palmitate, methylpropionate, and ethyl oleate; polyols and esters thereof such as propylene glycol, ethylene glycol, glycerol, butanediol, polyethylene glycol, and polyethylene glycol monolaurate (PEGML; see, e.g., U.S. Pat. No. 4,568,343); amides and other nitrogenous compounds such as urea, dimethylacetamide (DMA), dimethylformamide (DMF), 2-pyrrolidone, 1-methyl-2-pyrrolidone, ethanolamine, diethanolamine and triethanolamine; terpenes; alkanones; and organic acids, particularly salicylic acid and salicylates, citric acid, and succinic acid. The book Percutaneous Penetration Enhancers (Smith et al., editors, CRC Press, 1995) provides an excellent overview of the field and further background information on a number of chemical and physical enhancers.

Although many chemical permeation enhancers are known, there is an ongoing need for enhancers that (1) are highly effective in increasing the rate at which a drug permeates the skin; (2) do not result in skin damage, irritation, sensitization, or the like; and (3) can be used to effect dermal delivery of even high molecular weight drugs such as peptides, proteins, and nucleic acids. As the skin associated with many inflammatory dermatoses is especially difficult to penetrate, the topical treatment of such skin disorders would particularly benefit from more effective permeation enhancers. It has now been discovered that bases, for example, inorganic bases, such as hydroxide-releasing agents, and organic bases, such as amines and other nitrogenous bases, as well as other bases are highly effective permeation enhancers, even when used without co-enhancers, and provide all of the aforementioned advantages relative to known permeation enhancers. Furthermore, the permeation-enhancing bases of the invention are particularly effective in enhancing drug penetration into regions of skin affected by acne and other inflammatory dermatoses.

### SUMMARY OF THE INVENTION

It is accordingly a primary object of the invention to address the above needs in the art by providing a novel method and formulation for the treatment of inflammatory dermatoses, particularly acne vulgaris.

The invention provides a method and composition for the treatment of acne and other inflammatory dermatoses that involves a topically applied formulation containing a basic compound in an amount effective to provide the formulation with a pH in the range of about 8.0 to 13.0, plus an agent effective in treating acne or other inflammatory dermatoses. The formulation may be a lotion, cream, solution, paste, ointment, plaster, paint, bioadhesive, or the like, or may be contained in a tape or in a skin patch comprised of a laminated composite intended for long-term adhesion to the body surface (typically throughout a delivery period in the range of about 8 to about 72 hours) in the affected area.

In one aspect of the invention, a method is provided for enhancing the efficacy of an agent active in the treatment of acne or other inflammatory dermatoses by increasing the permeability of an affected area of the patient's body surface. The method involves administering the active agent to the affected area of the patient's body surface in combination with a permeation-enhancing base in a predetermined amount effective to enhance the flux of the agent through the body surface without causing damage thereto. The predetermined amount of the permeation-enhancing base is preferably an amount effective to provide a pH at the skin surface, i.e., during drug administration, in the range of about 8.0 to 13, preferably about 8.0 to 11.5, most preferably about 8.5 to 10.5. In another aspect, the pH is about 9.5 to 11.5, preferably about 10.0 to 11.5. If a skin patch is used, this is the preferred pH at the skin surface. The optimal amount (or concentration) of any one permeation-enhancing base will, however, depend on the specific base, i.e., on the strength or weakness of the base, its molecular weight, and other factors as will be appreciated by those of ordinary skill in the art of topical drug delivery. This optimal amount may be determined using routine experimentation to ensure that the pH at the skin surface is within the aforementioned ranges, i.e., in the range of about 8.0 to 13, preferably about 8.0 to 11.5, most preferably about 8.5 to 10.5. In some embodiments, the pH will be in the range of about 9.5 to 11.5, preferably about 10.0 to 11.5. A conventional transdermal drug delivery device or “patch” may be used to administer the active agent, in which case the drug and permeation-enhancing base are generally present in a drug reservoir or reservoirs. However, the drug and permeation-enhancing base may also be administered to the body surface using a liquid or semisolid formulation. Alternatively, or in addition, the body surface may be pretreated with the enhancer, e.g., treated with a dilute solution of the permeation-enhancing base prior to topical drug administration. Such a solution will generally be comprised of a protic solvent (e.g., water or alcohol) and have a pH in the range of about 8.0 to 13, preferably 8.0 to 11.5, and more preferably 8.5 to 10.5. As above, in some embodiments, the pH will be in the range of about 9.5 to 11.5, preferably about 10.0 to 11.5.

In a related aspect of the invention, a composition of matter is provided for delivering a drug effective for treating acne and other inflammatory dermatoses through a body surface using a basic compound as a permeation enhancer. Generally, the composition is a pharmaceutical formulation that comprises (a) a therapeutically effective amount of a drug effective in treating acne or another inflammatory dermatosis, (b) a basic compound in an amount effective to enhance the flux of the drug through the body surface without causing damage thereto, and (c) a pharmaceutically acceptable carrier suitable for topical drug administration. The composition may be in any form suitable for application to the body surface, and may comprise, for example, a cream, lotion, solution, gel, ointment, bioadhesive, paste, or the like, and/or may be prepared so as to contain liposomes, micelles, and/or microspheres. The composition may be directly applied to the body surface or may involve use of a drug delivery device. In one embodiment of the composition, the permeation-enhancing base is a hydroxide-releasing agent. In this embodiment, it is preferred although not essential that water be present in order for the hydroxide-releasing agent to generate hydroxide ions and thus enhance the flux of the active agent through the patient's body surface. Thus, a formulation or drug reservoir may be aqueous, i.e., contain water, or may be nonaqueous and used in combination with an occlusive overlayer so that moisture evaporating from the body surface is maintained within the formulation during drug administration.

In another aspect of the invention, a drug delivery system is provided for the topical administration of a drug effective in treating acne or other inflammatory dermatoses that uses a base as a permeation enhancer. The system will generally comprise: at least one drug reservoir containing the drug and the permeation-enhancing base in an amount effective to enhance the flux of the drug through the body surface without causing damage thereto; a means for maintaining the system in drug and enhancer transmitting relationship to the body surface; and a backing layer that serves as the outer surface of the device during use. The backing layer may be occlusive or nonocclusive, although it is preferably occlusive. The drug reservoir may be comprised of a polymeric adhesive, which may serve as the basal surface of the system during use and thus function as the means for maintaining the system in drug and enhancer transmitting relationship to the body surface. The drug reservoir may also be comprised of a hydrogel, or it may be a sealed pouch within a “patch”-type structure wherein the drug and permeation-enhancing base are present in the pouch as a liquid or semi-solid formulation.

### DETAILED DESCRIPTION OF THE INVENTION

I. Definitions and Nomenclature:

Before describing the present invention in detail, it is to be understood that this invention is not limited to specific permeation-enhancing bases, active agents, carriers, formulation types, treatment regimens, and so forth, as such may vary. It is also to be understood that the terminology used herein is for the purpose of describing particular embodiments only, and is not intended to be limiting.

It must be noted that, as used in this specification and the appended claims, the singular forms “a,” “an,” and “the” include plural referents unless the context clearly dictates otherwise.

The terms “permeation-enhancing base” and “basic compound” are used herein interchangeably to refer to a basic compound or composition of matter that is capable of producing a pH of 8.0 or greater in a pharmaceutically acceptable topical formulation. Such compositions include inorganic bases, such as hydroxide-releasing agents, and organic bases, such as amines and other nitrogenous bases.

The term “base” is used in its traditional sense, i.e., a substance that dissolves in water to produce hydroxide ions. The water is typically an aqueous fluid, and may be natural moisture at the skin surface, or the patch or composition that is used may contain added water, and/or be used in connection with an occlusive backing. Similarly, any liquid or semisolid formulation that is used is preferably aqueous or used in conjunction with an overlayer of an occlusive material. Any base may be used provided that the compound provides free hydroxide ions in the presence of an aqueous fluid. Bases can provide free hydroxide ions either directly or indirectly and thus can also be referred to as “hydroxide-releasing agents”. Hydroxide-releasing agents that provide free hydroxide ions directly, typically contain hydroxide groups and release the hydroxide ions directly into solution, for example, alkali metal hydroxides. Hydroxide-releasing agents that provide free hydroxide ions indirectly, are typically those compounds that are acted upon chemically in an aqueous environment and the reaction produces hydroxide ions, for example metal carbonates or amines.

“Active agent,” “pharmacologically active agent” and “drug” are used interchangeably herein to refer to a chemical material or compound that induces a desired pharmacological, physiological effect, and include agents that are therapeutically effective, prophylactically effective, or cosmeceutically effective. The terms also encompass pharmaceutically acceptable, pharmacologically active derivatives and analogs of those active agents specifically mentioned herein, including, but not limited to, salts, esters, amides, prodrugs, active metabolites, inclusion complexes, analogs, and the like. When the terms “active agent,” “pharmacologically active agent” and “drug” are used, then, it is to be understood that applicants intend to include the active agent per se as well as pharmaceutically acceptable, pharmacologically active salts, esters, amides, prodrugs, active metabolites, inclusion complexes, analogs, etc., which are collectively referred to herein as “pharmaceutically acceptable derivatives”. The term “active agent” is also intended to encompass “cosmeceutically active agents”, which are nontoxic agents that have medicinal or drug-like properties which, when applied to the surface of skin, beneficially affect the biological functioning of that skin.

By “pharmaceutically acceptable,” such as in the recitation of a “pharmaceutically acceptable carrier,” or a “pharmaceutically acceptable derivative,” is meant a compound that is not biologically or otherwise undesirable, i.e., the compound may be incorporated into a topical formulation of the invention and administered to a patient without causing any undesirable biological effects or interacting in a deleterious manner with any of the other components of the formulation in which it is contained.

The terms “treating” and “treatment” as used herein refer to actions that reduce the severity and/or frequency of symptoms, eliminate symptoms and/or their underlying cause, prevent the occurrence of symptoms and/or their underlying cause, and/or improve or remediate damage. The present method of “treating” a patient, as the term is used herein, thus encompasses both prevention of an inflammatory dermatosis in a predisposed individual and treatment of an inflammatory dermatosis in a clinically symptomatic individual.

The term “topical administration” is used in its conventional sense to mean delivery of a topical drug or pharmacologically active agent to the skin or mucosa. Topical administration, in contrast to transdermal administration, provides exclusively or predominantly a local rather than a systemic effect. The term “transdermal” is intended to include “transmucosal” drug administration, i.e., administration of a drug to the mucosal (e.g., sublingual, buccal, vaginal, rectal) surface of an individual so that the drug passes through the mucosal tissue and into the individual's blood stream.

The term “body surface” is used to refer to skin or mucosal tissue.

By “predetermined area” of skin or mucosal tissue, which refers to the area of skin or mucosal tissue through which a formulation of the invention is delivered, is meant a defined area of living skin or mucosal tissue affected by acne or another inflammatory dermatosis.

“Penetration enhancement” or “permeation enhancement” as used herein relates to an increase in the permeability of the skin or mucosal tissue to the selected pharmacologically active agent, i.e., so that the rate at which the agent permeates therethrough (i.e., the “flux” of the agent through the body surface) is increased relative to the rate that would be obtained in the absence of permeation enhancer. The enhanced permeation effected through the use of such enhancers can be observed by measuring the rate of diffusion of drug through animal or human skin using, for example a Franz diffusion apparatus as known in the art and as employed in the Examples herein.

“Effective amount” or “an effective permeation enhancing amount” of a permeation enhancer refers to a nontoxic, non-damaging but sufficient amount of the enhancer composition to provide the desired increase in skin permeability and, correspondingly, the desired depth of penetration, rate of administration, and amount of drug delivered.

By an “effective” amount or a “therapeutically effective amount” of a pharmacologically active agent is meant a nontoxic but sufficient amount of the drug or agent to provide the desired effect, i.e., prevention or treatment of acne or other inflammatory dermatoses. The amount that is “effective” will vary from subject to subject, and it is not always possible to specify an exact “effective amount.” However, an appropriate “effective” amount in any individual case may be determined by one of ordinary skill in the art using routine experimentation.

“Carriers” or “vehicles” as used herein refer to carrier materials suitable for topical drug administration. Carriers and vehicles useful herein include any such materials known in the art that are nontoxic and do not interact with other components of the composition in a deleterious manner.

The term “aqueous” refers to a formulation or drug delivery system that contains water or that becomes water-containing following application to the skin or mucosal tissue.

In describing molecular structures and formulae herein, the phrase “having the formula” or “having the structure” is not intended to be limiting and is used in the same way that the term “comprising” is commonly used.

The term “alkyl” as used herein refers to a branched or unbranched saturated hydrocarbon group typically although not necessarily containing 1 to about 24 carbon atoms, such as methyl, ethyl, n-propyl, isopropyl, n-butyl, isobutyl, t-butyl, octyl, decyl, and the like, as well as cycloalkyl groups such as cyclopentyl, cyclohexyl, and the like. Generally, although again not necessarily, alkyl groups herein contain 1 to about 12 carbon atoms. If not otherwise indicated, the term “alkyl” includes linear, branched, cyclic, unsubstituted, substituted, and/or heteroatom-containing alkyl.

The term “alkenyl” as used herein refers to a branched or unbranched hydrocarbon group typically although not necessarily containing 2 to about 24 carbon atoms and at least one double bond, such as ethenyl, n-propenyl, isopropenyl, n-butenyl, isobutenyl, octenyl, decenyl, and the like. Generally, although again not necessarily, alkenyl groups herein contain 2 to about 12 carbon atoms.

The term “aryl” as used herein, and unless otherwise specified, refers to an aromatic substituent. Preferred aryl groups contain one aromatic ring and are referred to as “monocyclic aryl.”

The terms “alkyl,” “alkenyl,” “aryl,” and the like are, unless otherwise indicated, intended to include unsubstituted, substituted, heteroatom-containing, and substituted heteroatom-containing substituents.

Accordingly, the invention pertains to a method, composition, and drug delivery system for enhancing the efficacy of a topically applied active agent in the treatment of acne or other inflammatory dermatoses by increasing the rate at which the active agent permeates into the body surface of a patient, wherein the method involves administering the active agent to a predetermined area of the patient's body surface in combination with a permeation-enhancing base in an amount effective to enhance the flux of the active agent through the body surface without causing damage thereto.

II. Indications:

The invention pertains to treatment of an individual predisposed to or afflicted with an inflammatory dermatosis, comprising topically administering to the individual's affected skin area a pharmaceutical formulation containing a therapeutically effective amount of an agent active for treating an inflammatory dermatosis, together with a permeation-enhancing amount of a base, wherein the formulation has a pH in the range of about 8.0 to about 13.0, preferably about 8.0 to 11.5, and most preferably about 8.5 to 11.0. In some embodiments, the pH will be in the range of about 9.5 to 11.5, preferably about 10.0 to 11.5. The term “inflammatory dermatosis” includes a range of skin disorders, including, but not limited to, sebaceous gland disorders, papulosquamous dermatoses, allergic dermatoses, pruritic dermatoses, vascular dermatoses, bacterial dermatoses, viral dermatoses, mycolic skin infections, granulomatous dermatoses, parasitic skin dermatoses, exfoliative dermatitis, bullous dermatoses, pigmented dermatoses, photosensitive dermatoses, dermatoses caused by collagen diseases, and dermatoses due to internal diseases. The inflammatory dermatosis can also be associated with an autoimmune condition, in which case it is referred to herein as “autoimmune dermatosis.”

In a preferred embodiment, the inflammatory dermatosis that the method and formulation of the invention are used to treat is a sebaceous gland disorder, e.g., an acneiform disorder such as acne vulgaris, acne conglombata, hidradenitis suppurativa, acne rosacea, seborrhea, seborrheic dermatitis, gram negative folliculitis, pyoderma faciale, steatocystoma multiplex, sebaceous hyperplasia, or rhinophyma. In a particularly preferred embodiment, the invention is used to treat acne vulgaris. As is well known, acne vulgaris is a chronic skin condition characterized by comedones and papules, and can be quite severe; in particularly severe cases, pustules, cysts, and permanent scarring may occur.

In another preferred embodiment, the inflammatory dermatosis treated is a papulosquamous dermatosis such as, for example, psoriasis, Pityriasis rosea, tinea versicolor, or lichen planus. The method and formulations of the invention are particularly useful in treating psoriasis, an autoimmune inflammatory disorder that has proven difficult to treat with conventional agents.

In a further preferred embodiment, the inflammatory dermatosis treated is an autoimmune dermatosis that may be, by way of example, atopic dermatitis, mast cell disease, bullous pemphigoid, pemphigus vulgaris, necrotizing vasculitis, discoid lupus erytbematosus, systemic lupus erythematosis, or dermatitis herpetiformis.

In other embodiments, examples of the various types of inflammatory dermatoses with which the method and formulation of the invention are effective are as follows:

Allergic Dermatoses: contact dermatitis; photoallergic dermatitis; industrial dermatoses caused by exposure to a variety of compounds used by industry that are contact irritants; atopic eczema (infantile and adult); and dermatoses caused by drugs and nummular eczema.

Pruritic Dermatoses: winter, senile, and essential pruritus; pruritus ani; eternal otitis; pruritis hiemalis; pruritis vulvae; and pruritus scrotae.

Vascular Dermatoses: erythema multiforme; erythema nodosum; stasis dermatitis; purpuric dermatoses such as those associated with thrombocytopenic purpura, nonthrombocytopenic purpura, dysproteinemic purpura, actinic purpura, scorbutic purpura, and Henochs purpura; ecchymoses; stasis purpura; primary and secondary telangiectases.

Bacterial Dermatoses: pyoderma such as impetigo, ecthyma, folliculitis, furuncles styes, carbuncles, sweat gland infections, erysipelas, erythrasma, infected ulcers, and infected eczematoid dermatitis; and bacterial dermatoses associated with systemic bacterial infections such as scarlet fever, granuloma inguinale, chancroid, tuberculosis, leprosy, gonorrhea, rickettsial diseases, actinomycosis, and syphilis.

Viral Dermatoses: such as those caused by herpes simplex virus, Kaposi's varicelliform eruption, zoster, chickenpox, smallpox, vaccinia, molluscum contagiosum, lymphogranuloma venereum, exanthematous diseases such as German measles, roseola, and erythema infectiosum.

Mycolic Skin Infections: tinea cruris (superficial fungal infections of the skin in various body sites); athlete's foot (dermatophytosis of the feet caused to infection with trichophyton mentagrophytes); tinea unguium (onychomycosis); sporotrichosis; coccidioidomycosis; histoplasmosis; and North American blastomycosis.

Granulomatous Dermatoses: sarcoidosis; granuloma annulare; reticulohistiocytoma; and silica-induced granulomas.

Parasitic Skin Infections: scabies, cheyletiella dermatitis; demodicosis; pediculosis

Pigmented Dermatoses: Chloasma (melasma) and vitiligo.

Collagen Diseases: scleroderma and dermatomyositis.

Dermatoses Due to Internal Diseases: pyoderma gangrenosum associated with ulcerative colitis, and ulcers due to diabetes.

Photosensitive Dermatoses: Exogenous types such as drug-induced photodermatitis and contact dermatitis with photoallergic components; and endogenous types such as those associated with porphyrias and polymorphous light eruptions.

III. The Permeation Enhancers:

The permeation enhancer of the invention is an inorganic or an organic pharmaceutically acceptable base. Preferred inorganic bases include inorganic hydroxides, inorganic oxides, inorganic salts of weak acids, and combinations thereof. Preferred organic bases are nitrogenous bases.

It has long been thought that strong bases, such as NaOH, were not suitable as permeation enhancers because they would damage skin. It has been now been discovered that the skin permeability of various drugs could be enhanced without skin damage by exposing the skin to a base or basic solution, in a skin contacting formulation or patch. The desired pH of the solution on the skin can be obtained using a variety of bases or base concentrations. Accordingly, the pH is selected so as to be low enough so as to not cause skin damage, but high enough to enhance skin permeation to various active agents. As such, it is important that the amount of base in any patch or formulation is optimized so as to increase the flux of the drug through the body surface while minimizing any possibility of skin damage. In general, this means that the pH at the body surface in contact with a formulation or drug delivery system of the invention is preferably in the range of approximately 8.0-13.0, preferably about 8.0-11.5, more preferably about 8.5 to 11.5 and most preferably about 8.5-10.5. In some embodiments, the pH will be in the range of about 9.5 to 11.5, preferably about 10.0 to 11.5.

In one preferred embodiment, the pH at the skin surface is the primary design consideration, i.e., the composition or system is designed so as to provide the desired pH at the skin surface. Anhydrous formulations and transdermal systems may not have a measurable pH, and the formulation or system can be designed so as to provide a target pH at the skin surface. Moisture from the body surface can migrate into the formulation or system, dissolve the base and thus release the base into solution, which will then provide the desired target pH at body surface. In those instances, a hydrophilic composition is preferred. In addition, when using aqueous formulations, the pH of the formulation may change over time after it is applied on the skin. For example, gels, solutions, ointments, etc., may experience a net loss of moisture after being applied to the body surface, i.e., the amount of water lost is greater than the amount of water received from the body surface. In that case, the pH of the formulation may be different than its pH when manufactured. This problem can be easily remedied by designing the aqueous formulations to provide a target pH at the body surface.

In other embodiments of the invention, the pH of the formulation or the drug composition contained within a delivery system will be in the range of approximately 8.0 to 13.0, preferably about 8.0 to 11.5, more preferably about 8.5 to 11.5, and most preferably about 8.5 to 10.5. In some embodiments, the pH will be in the range of about 9.5 to 11.5, preferably about 10.0 to 11.5. In one embodiment of the invention the pH of the formulation is higher than the pH at the body surface. For example, if an aqueous formulation is used, moisture from the body surface can dilute the formulation, and thus provide for a different pH at the body surface, which will typically be lower than that of the formulation itself.

In one preferred embodiment, the body surface is exposed to a base or basic solution for a sufficient period of time so as to provide a high pH at the skin surface, thus creating channels in the skin or mucosa for the drug to go through. It is expected that drug flux is proportional to the strength of the solution and the duration of exposure. However, it is desirable to balance the maximization of drug flux with the minimization of skin damage. This can be done in numerous ways. For example, the skin damage may be minimized by selecting a lower pH within the 8.0 to 13.0 range, by exposing the skin to the formulation or system for a shorter period of time, or by including at least one irritation-mitigating additive. Alternatively, the patient can be advised to change the location of application with each subsequent administration.

While certain preferred amounts are set forth below, it is understood that, for all of the inorganic and organic bases described herein, the optimum amount of any such base will depend on the strength or weakness of the base and its molecular weight, and other factors such as the number of ionizable sites in the active agent being administered and whether there are any acidic species present in the formulation or patch. One skilled in the art may readily determine the optimum amount for any particular base such that the degree of enhancement is optimized while the possibility of damage to the body surface is eliminated or at least substantially minimized.

Inorganic Base

Exemplary inorganic bases are inorganic hydroxides, inorganic oxides, inorganic salts of weak acids, and combinations thereof. Preferred inorganic bases are those whose aqueous solutions have a high pH, and are acceptable as food or pharmaceutical additives. Examples of such preferred inorganic bases are those listed below, along with their respective pHs. Some of the bases are identified by their hydrate forms, and it is understood that when referring to a “base”, both the hydrated and non-hydrated forms are intended to be included.

| | pH of Aqueous | |
|---|---|---|
| Inorganic base | Solution (concentration) | |
| Ammonium hydroxide^{1,2,3} | 11.27 (1 N), 10.27 (0.001 N) | |
| Sodium hydroxide^{1,2,3} | 14 (5%), 13 (0.5%), 12 (0.05%) | |
| Potassium hydroxide^{1,2,3} | 13.5 | (0.1 M) |
| Calcium hydroxide^{1,3} | 12.4 | (saturated solution |
| | | in water) |
| Magnesium hydroxide^{1,3} | 9.5 to 10.5 | slurry |
| Magnesium oxide^{1,2,3} | 10.3 | (saturated aqueous |
| | | solution) |
| Calcium oxide³ | Soluble in water, Form Ca(OH)₂ | |
| Sodium acetate^{1,3} | ˜89 | (0.1 N) |
| Sodium acetate, trihydrate^{1,2} | 8.9 | (0.1 N) |
| Sodium acetate, anhydrous^{1,2} | ˜8.9 | (0.1 N) |
| Sodium borate decahydrate^{1,2} | ˜8.8-9.4, | (0.01 M) |
| | 9.15 to 9.2 | |
| Sodium borate^{1,2,3} | 8.8-9.4, | (0.01 M) |
| | 9.15 to 9.2 | |
| Sodium metaborate | Strongly alkaline | |
| Sodium carbonate^{1,2,3} | ˜11.6 | |
| Sodium carbonate hydrate¹ | ˜11.6 | |
| Sodium carbonate anhydrous | ˜11.6 | |
| Sodium bicarbonate^{1,2,3} | 8.3 | (0.1 M fresh) |
| Sodium phosphate, tribasic^{1,3} | ˜11.5 (0.1%), ˜11.7 (0.5%), | |
| | ˜11.9 | (1.0%) |
| Sodium phosphate, tribasic | 11.5 (0.1%), 11.7 (0.5%), | |
| dodecahydrate | 11.9 | (1.0%) |
| Sodium phosphate, dibasic, | 9.1 | (1%) |
| anhydrous^{1,2} | | |
| Sodium phosphate, dibasic, | ˜9.5 | |
| heptahydrate^{1,2} | | |
| Sodium phosphate, dibasic^{1,3} | ˜9.5 | |
| Sodium phosphate, dibasic, | ˜9.5 | |
| dihydrate¹ | | |
| Sodium phosphate, dibasic, | ˜9.5 | |
| dodecahydrate | | |
| Potassium carbonate^{1,3} | ˜11.6 | |
| Potassium bicarbonate³ | 8.2 | (0.1 M) |
| Potassium citrate^{1,2,3} | ˜8.5 | |
| Potassium citrate monohydrate | ˜8.5 | |
| Potassium acetate^{1,3} | 9.7 | (0.1 M) |
| Potassium phosphate, dibasic^{1,2} | Aqueous solution is slightly | |
| | alkaline | |
| Potassium phosphate, tribasic³ | Aqueous solution is strongly | |
| | alkaline | |
| Ammonium phosphate, dibasic^{1,2,3} | ˜8 | |

¹listed in the “Chemicals in Compliance with Pharmaceutical Standards: Inactive Ingredient Guide”
²listed in the “Handbook of Pharmaceutical Additives”
³listed in the FDA's food additive database

### Inorganic Hydroxides

Inorganic hydroxides include, for example, ammonium hydroxide, alkali metal hydroxide and alkaline earth metal hydroxides, and mixtures thereof. Preferred inorganic hydroxides include ammonium hydroxide; monovalent alkali metal hydroxides such as sodium hydroxide and potassium hydroxide; divalent alkali earth metal hydroxides such as calcium hydroxide and magnesium hydroxide; and combinations thereof.

The amount of inorganic hydroxide included in the compositions and systems of the invention, will typically represent about 0.3-7.0 wt %, preferably 0.5-4.0 wt %, more preferably about 0.5-3.0 wt %, most preferably about 0.75-2.0 wt %, of a topically applied formulation or of a drug reservoir of a drug delivery system, or patch.

The aforementioned amounts are particularly applicable to those formulations and patches in which the active agent is (1) an uncharged molecule, e.g., wherein a basic drug is in nonionized, free-base form, (2) a basic salt of an acidic drug, or (3) there are no additional species in the formulation or patch that could react with or be neutralized by the inorganic hydroxide, to any significant degree.

For formulations and patches in which the drug is in the form of an acid addition salt, and/or wherein there are additional species in the formulations or systems that can be neutralized by or react with the inorganic base (i.e., acidic inactive ingredients), the amount of inorganic hydroxide is preferably the total of (1) the amount necessary to neutralize the acid addition salt and/or other base-neutralizable species (i.e., the “acidic species”), plus (2) about 0.3-7.0 wt %, preferably 0.5-4.0 wt %, more preferably about 0.5-3.0 wt %, most preferably about 0.75-2.0 wt %, of the formulation or drug reservoir. That is, for an acid addition salt, the enhancer is preferably present in an amount just sufficient to neutralize the salt, plus an additional amount (i.e., about 0.3-7.0 wt %, preferably 0.5-4.0 wt %, more preferably about 0.5-3.0 wt %, most preferably about 0.75-2.0 wt %) to enhance the flux of the drug through the skin or mucosal tissue. Basic drugs in the form of a neutral, free base or basic salt of acidic drug are usually not affected by a base, and thus for these drugs, the amount in (1) is usually the amount necessary to neutralize inactive components that are acidic. For patches, the aforementioned percentages are given relative to the total weight of the formulation components and the adhesive, gel or liquid reservoir.

Still greater amounts of inorganic hydroxide may be used by controlling the rate and/or quantity of release of the base, preferably during the drug delivery period itself.

Inorganic Oxides

Inorganic oxides include, for example, magnesium oxide, calcium oxide, and the like.

The amount of inorganic oxide included in the compositions and systems of the invention may be substantially higher than the numbers set forth above for the inorganic hydroxide, and may be as high as 20 wt %, in some cases as high as 25 wt % or higher, but will generally be in the range of about 2-20 wt %. These amounts may be adjusted to take into consideration the presence of any base-neutralizable species.

### Inorganic Salts of Weak Acids

Inorganic salts of weak acids include, ammonium phosphate (dibasic); alkali metal salts of weak acids such as sodium acetate, sodium borate, sodium metaborate, sodium carbonate, sodium bicarbonate, sodium phosphate (tribasic), sodium phosphate (dibasic), potassium carbonate, potassium bicarbonate, potassium citrate, potassium acetate, potassium phosphate (dibasic), potassium phosphate (tribasic); alkaline earth metal salts of weak acids such as magnesium phosphate and calcium phosphate; and the like, and combinations thereof.

Preferred inorganic salts of weak acids include, ammonium phosphate (dibasic) and alkali metal salts of weak acids.

The amount of inorganic salts of weak acids included in the compositions and systems of the invention may be substantially higher than the numbers set forth above for the inorganic hydroxide, and may be as high as 20 wt %, in some cases as high as 25 wt % or higher, but will generally be in the range of approximately 2-20 wt %. These amounts may be adjusted to take into consideration the presence of any base-neutralizable species.

Organic Bases

Organic bases suitable for use in the invention are compounds having an amino group, amido group, an oxime, a cyano group, an aromatic or non-aromatic nitrogen-containing heterocycle, a urea group, and combinations thereof. More specifically, examples of suitable organic bases are nitrogenous bases, which include, but are not limited to, primary amines, secondary amines, tertiary amines, amides, oximes, cyano (—CN) containing groups, aromatic and non-aromatic nitrogen-containing heterocycles, urea, and mixtures thereof. Preferred organic bases are primary amines, secondary amines, tertiary amines, aromatic and non-aromatic nitrogen-containing heterocycles, and mixtures thereof.

For nitrogenous bases, the amount of enhancing agent will typically represent about 0.5-4.0 wt %, preferably about 0.5-3.0 wt %, more preferably about 0.75-2.0 wt %, of a topically applied formulation or of a drug reservoir of a drug delivery system or a patch. These amounts may be adjusted to take into consideration the presence of any base-neutralizable species.

Still greater amounts of the nitrogenous base may be used depending on the strength of the base and the rate and/or quantity of release of the nitrogenous base preferably during the drug delivery period itself.

Preferred organic bases are those whose aqueous solutions have a high pH or a high pKa (more preferably a pKa>9), and are acceptable as food or pharmaceutical additives. Examples of such preferred organic bases are those listed below, along with their respective pHs (or pKa values).

| | pH of Aqueous | |
|---|---|---|
| Organic base | Solution (concentration) | |
| 2-amino-2-methyl-1,3-propanediol¹ | 10.8 | (0.1 M) |
| 2-amino-2-methyl-1-propanol¹ | 11.3 | (0.1 M) |
| Diethanolamine¹ | 11.0 | (0.1 N) |
| Triethanolamine¹ | 10.5 | (0.1 N) |

| | |
|---|---|
| Butylamine² | pKa = 10.56 |
| Dimethylamine² | Strong base, pKa = 10.73 |
| Cyclohexylamine² | Strong base, pKa = 10.64 |
| Ethylenediamine² | Strong base, pKa = 10.71 |
| Isopentylamine² | pKa = 10.6 |
| Monoethanolamine² | 12.1 (25%), 12.05 (0.1 N), pKa = 9.4 |
| Phenethylamine² | Strong base, pKa = 9.83 |
| Piperidine² | Strong base, pKa = 11.12 |
| Pyrrolidine² | Strong base, pKa = 11.27 |
| Trimethylamine² | Strong base, pKa = 9.81 |

¹listed in the “Handbook of Pharmaceutical Additives”
²listed in the FDA's food additive database

### Amines

Specific nitrogenous bases may contain any one or a combination of the following:

primary amino (—NH₂) groups;

mono-substituted (secondary) amino groups —NHR where R is hydrocarbyl, generally either alkyl or aryl, e.g., lower alkyl or phenyl, and may be substituted with one or more nonhydrocarbyl substituents, e.g., 1 to 3 halo, hydroxyl, thiol, or lower alkoxy groups (such —NHR groups include, for example, methylamino, ethylamino, isopropylamino, butylamino, cyclopropylamino, cyclohexylamino, n-hexylamino, phenylamino, benzylamino, chloroethylamino, hydroxyethylamino, etc.);

di-substituted (tertiary) amino groups —NR^{a}R^{b} where R^{a} and R^{b} may be the same or different and are as defined above for R (suitable —NR^{a}R^{b} include, for example, dimethylamino, diethylamino, diisopropylamino, dibutylamino, methylpropylamino, methylhexylamino, methylcyclohexylamino, ethylcyclopropylamino, ethylchloroethylamino, methylbenzylamino, methylphenylamino, methyltoluylamino, methyl-p-chlorophenylamino, methylcyclohexylamino, etc.);

amides —(CO)—NR^{c}R^{d} where R^{c} and R^{d} may be the same or different and are either hydrogen or R, wherein R is as defined above (including, for example, amides wherein one of R^{c} and R^{d} is H and the other is methyl, butyl, benzyl, etc.);

cyano (—CN);

aromatic nitrogen-containing heterocycles, typically five- or six-membered monocyclic substituents, or bicyclic fused or linked five- or six-membered rings (such as pyrrolyl, pyrrolidinyl, pyridinyl, quinolinyl, indolyl, pyrimidinyl, imidazolyl, 1,2,4-triazolyl, tetrazolyl, etc.); and

non-aromatic nitrogen-containing heterocycles, typically four- to six-membered rings, including lactams and imides, e.g., pyrrolidino, morpholino, piperazino, piperidino, N-phenyl—propiolactam, -butyrolactam, -caprolactam, acetimide, phthalimide, succinimide, etc.

Primary amines, secondary amines, and tertiary amines may be generically grouped as encompassed by the molecular structure NR¹R²R³ wherein R¹, R² and R³ are selected from H, alkyl, hydroxyalkyl, alkoxyalkyl, alkenyl, hydroxyalkenyl, alkoxyalkenyl, cycloalkyl, cycloalkyl-substituted alkyl, monocyclic aryl, and monocyclic aryl-substituted alkyl, with the proviso that at least one of R¹, R² and R³ is other than H. Examples of such amines include, without limitation, diethanolamine, triethanolamine, isopropanolamine, triisopropanolamine, dibutanol amine, tributanol amine, N-dodecylethanolamine, N-(2-methoxyethyl) dodecylamine, N-(2,2-dimethoxyethyl)dodecylamine, N-ethyl-N-(dodecyl)ethanolamine, N-ethyl-N-(2-methoxyethyl)dodecylamine, N-ethyl-N-(2,2-dimethoxyethyl) dodecylamine, dimethyldodecylamine-N-oxide, monolauroyl lysine, dipalmitoyl lysine, dodecylamine, stearylamine, phenylethylamine, triethylamine, PEG-2 oleamine, PEG-5 oleamine, dodecyl 2-(N,N-dimethylamino)propionate, bis(2-hydroxyethyl)oleylamine, and combinations thereof.

Exemplary primary amines include 2-aminoethanol, 2-aminoheptane, 2-amino-2-methyl-1,3 propanediol, 2-amino-2-methyl-1-propanol, n-amylamine, benzylamine, 1,4-butanediamine, n-butylamine, cyclohexylamine, ethylamine, ethylenediamine, methylamine, α-methylbenzylamine, phenethylamine, propylamine, and tris(hydroxymethyl)aminomethane.

Exemplary secondary amines include compounds that contain groups such as methylamino, ethylamino, isopropylamino, butylamino, cyclopropylamino, cyclohexylamino, n-hexylamino, phenylamino, benzylamino, chloroethylamino, hydroxyethylamino, and so forth. Exemplary secondary amines include diethanolamine, diethylamine, diisopropylamine, and dimethylamine.

Exemplary tertiary amines include compounds that contain groups such as dibutylamino, diethylamino, dimethylamino, diisopropylamino, ethylchloroethylamino, ethylcyclopropylamino, methylhexylamino, methylcyclohexylamino, methylpropylamino, methylbenzylamino, methyl-p-chlorophenylamino, methylcyclohexylamino, methylphenylamino, methyltoluylamino, and so forth. Exemplary tertiary amines include N,N-diethylaniline, N,N-dimethylglycine, triethanolamine, triethylamine, and trimethylamine.

### Amides

Amides, as will be appreciated by those skilled in the art, have the molecular structure R⁴—(CO)—NR⁵R⁶ where R⁴, R⁵ and R⁶ are generally selected from H, alkyl, cycloalkyl, cycloalkyl-substituted alkyl, monocyclic aryl, and monocyclic aryl-substituted alkyl. Examples of suitable amides herein include, without limitation, hexamethyleneacetamide, hexamethyleneoctamide, hexamethylene lauramide, hexamethylene palmitamide, N,N-dimethyl formamide, N,N-dimethyl acetamide, N,N-dimethyloctamide, N,N-dimethyidecamide, toluamide, dimethyl-m-toluamide, diethyl-m-toluamide, and combinations thereof.

### Nitrogen-Containing Heterocycles

Nitrogen-containing heterocycles suitable as the pharmacologically active base herein include, by way of example, 2-pyrrolidone, 1-methyl-2-pyrrolidone, 5-methyl-2-pyrrolidone, 1,5-dimethyl-2-pyrrolidone, 1-ethyl-2-pyrrolidone, 1-propyl-3-dodecylpyrrolidine, 1-dodecyclazacycloheptan-2-one, ethylene thiourea, hydantoin, oxalylurea, imidazolidilyl urea, N-octadecyl morpholine, dodecylpyridinium, N-dodecylpyrrolidine, N-dodecylpiperidine, N-dodecylhomopiperidine, and combinations thereof.

Aromatic nitrogen-containing heterocycles, typically contain a 5- or 6-membered monocyclic substituent, or a bicyclic fused or linked 5- or 6-membered ring, such as imidazolyl, indolyl, pyridinyl, pyrimidinyl, pyrrolyl, quinolinyl, tetrazolyl, 1,2,4-triazolyl, etc.

Aromatic nitrogen-containing heterocycles suitable as the organic base herein include, by way of example, 2-amino-pyridine, benzimidazole, 2,5-diaminopyridine, 2,4-dimethylimidazole, 2,3-dimethylpyridine, 2,4-dimethylpyridine, 3,5-dimethylpyridine, imidazole, methoxypyridine, γ-picoline, 2,4,6-trimethylpyridine, and combinations thereof.

Non-aromatic nitrogen-containing heterocycles, typically contain 4- to 6-membered rings such as acetimido, morpholinyl, lactams and imides (e.g., -butyrolactam, -caprolactam, N-phenyl-propiolactam), phthalimido, piperidyl, piperidino, piperazinyl, pyrrolidinyl, succinimido, etc.

Non-aromatic nitrogen-containing heterocycles include, by way of example, 1,2-dimethylpiperidine, 2,5-dimethylpiperazine, 1,2-dimethylpyrrolidine, 1-ethylpiperidine, n-methylpyrrolidine, morpholine, piperazine, piperidine, pyrrolidine, 2,2,6,6-tetramethylpiperidine, 2,2,4-trimethylpiperidine, and combinations thereof.

For all permeation-enhancing bases herein, the optimum amount of any particular agent will depend on the strength or weakness of the base, the molecular weight of the base, and other factors such as the number of ionizable sites in the drug administered and any other acidic species in the formulation or patch. One skilled in the art may readily determine the optimum amount for any particular agent by ensuring that a formulation is effective to provide a pH at the skin surface, upon application of the formulation, in the range of about 7.5 to about 13.0, preferably about 8.0 to about 11.5, preferably in the range of about 8.5 to about 10.5. In some embodiments, the pH will be in the range of about 9.5 to 11.5, preferably about 10.0 to 11.5. This in turn ensures that the degree of treatment is maximized while the possibility of damage to the body surface is eliminated or at least substantially minimized.

IV. The Active Agent:

The active agent may be any compound or other composition of matter that is suitable for topical delivery, is effective in the treatment of warts, and is compatible with the permeation-enhancing base. Active agents of the invention thus include, without limitation, keratolytic compounds such as salicylic acid; proinflammatory agents such as imiquimod; contact sensitizers such as dinitrochlorobenzene and dibutyl squaric acid; irritants and blistering agents such as podophyllin, podophyllotoxin, cantharidin, and trichloroacetic acid; retinoids such as tretinoin (all-trans-retinoic acid); antiproliferative agents such as 5-fluorouracil; bleomycin and related compounds; interferon-alpha; alpha hydroxy acids such as lactic acid and glycolic acid; alpha keto acids such as glyoxylic acid and the alpha keto acids discussed, for example, in U.S. Pat. No. 5,674,899; and antiviral agents. Antiviral agents are preferred, and include, without limitation: nucleoside phosphonates and other nucleoside analogs, such as cidofovir [(S)-1-(3-hydroxy-2-phosphonylmethoxypropyl)cytosine] (HPMPC), cyclic HPMPC (cHPMPC), adefovir [9-((2-phosphonylmethoxy)ethyl)adenine] (PMEA), and cyclopropyl PMEDAP [cyclopropyl 9-(2-phosphonylmethoxyethyl)-2,6- diaminopurine]; AICAR (5-amino-4-imidazolecarboxamide ribonucleotide) analogs such as ribavirin; glycolytic pathway inhibitors, such as 2-deoxyglucose; and anionic polymers and polysaccharide polymers such as polysulfates (e.g., PVAS), polysulfonates (e.g., polystyrene sulfonate, polyvinyl sulfonate), polycarboxylates, polyoxometalates, cellulose sulfate, dextran sulfate, chicoric acid, zintevir, and cosalane derivatives. Nucleoside phosphonates are particularly preferred, especially cidofovir.

Combinations of one or more agents active in treating warts are also included within the scope of the invention. Any such combinations that are compatible with each other and with the permeation-enhancing base are included.

V. Pharmaceutical Formulations and Skin Patches:

The pharmaceutical formulation of the invention comprises one or more active agents and a permeation-enhancing base as described above, in a pharmaceutically acceptable topical carrier. The permeation-enhancing base is present at a concentration sufficient to provide a formulation pH in the range of approximately 8.0 to 13.0, preferably 8.0 to 11.5, most preferably 8.5 to 10.5.0. The active agent or agents are present at a concentration effective to treat the inflammatory dermatosis for which treatment is desired.

The particular combination of active agent or agents and permeation-enhancing base is determined in large part by chemical compatibility. That is, each active agent must coexist in the topical pharmaceutical formulation together with the base and any other active agent without reacting or otherwise interacting with each other or with other components of the formulation in a way that would diminish therapeutic efficacy or increase the likelihood of toxic or other adverse effects. Thus, for example, direct contact between a strong inorganic base, such as potassium hydroxide, and an acid, such as salicylic acid, should be avoided, as such compounds may react with each other in deleterious ways. Even such reactive pairs of compounds may, however, be combined in an effective topical formulation if; for example, the active agent is contained within liposomes; micelles, microspheres, or similar structures, so that it is released after permeation into the skin and after the base has dissipated sufficiently to avoid significant reaction with the active agent.

The formulation may be in any form suitable for application to the body surface, such as a cream, lotion, solution, gel, ointment, paste, plaster, paint, bioadhesive, or the like, and/or may be prepared so as to contain liposomes, micelles, and/or microspheres. For those formulations in which the permeation-enhancing base is an inorganic base, such as a hydroxide-releasing agent, it is preferred although not essential that water be present. Thus, such a formulation may be aqueous, i.e., contain water, or may be nonaqueous and optionally used in combination with an occlusive overlayer so that moisture evaporating from the body surface is maintained within the formulation upon application to the body surface and thereafter.

Formulations of the invention may optionally contain a pharmaceutically acceptable viscosity enhancer and/or film former. A viscosity enhancer increases the viscosity of the formulation so as to inhibit its spread beyond the site of application. Balsam Fir (Oregon) is an example of a pharmaceutically acceptable viscosity enhancer.

A film former, when it dries, forms a protective film over the site of application. The film inhibits removal of the active ingredient and keeps it in contact with the site being treated. An example of a film former that is suitable for use in this invention is Flexible Collodion, USP. As described in Remington: The Science and Practice of pharmacy, 19th Ed. (Easton, Pa: Mack Publishing Co., 1995), at page 1530, collodions are ethyl ether/ethanol solutions containing pyroxylin (a nitrocellulose) that evaporate to leave a film of pyroxylin. A film former may act additionally as a carrier. Solutions that dry to form a film are sometimes referred to as paints.

Ointments, as is well known in the art of pharmaceutical formulation, are semisolid preparations that are typically based on petrolatum or other petroleum derivatives. The specific ointment base to be used, as will be appreciated by those skilled in the art, is one that will provide for optimum drug delivery, and, preferably, will provide for other desired characteristics as well, e.g., emolliency or the like. As with other carriers or vehicles, an ointment base should be inert, stable, nonirritating and nonsensitizing. As explained in Remington: The Science and Practice of Pharmacy, 19th Ed. (Easton, Pa.: Mack Publishing Co., 1995), at pages 1399-1404, ointment bases may be grouped in four classes: oleaginous bases; emulsifiable -bases; emulsion bases; and water-soluble bases. Oleaginous ointment bases include, for example, vegetable oils, fats obtained from animals, and semisolid hydrocarbons obtained from petroleum. Emulsifiable ointment bases, also known as absorbent ointment bases, contain little or no water and include, for example, hydroxystearin sulfate, anhydrous lanolin and hydrophilic petrolatum. Emulsion ointment bases are either water-in-oil (W/O) emulsions or oil-in-water (O/W) emulsions, and include, for example, cetyl alcohol, glyceryl monostearate, lanolin, and stearic acid. Preferred water-soluble ointment bases are prepared from polyethylene glycols of varying molecular weight; again, see Remington: The Science and Practice of Pharmacy for further information.

Creams, as also well known in the art, are viscous liquids or semisolid emulsions, either oil-in-water or water-in-oil. Cream bases are water-washable, and contain an oil phase, an emulsifier, and an aqueous phase. The oil phase, also called the “internal” phase, is generally comprised of petrolatum and a fatty alcohol such as cetyl or stearyl alcohol. The aqueous phase usually, although not necessarily, exceeds the oil phase in volume, and generally contains a humectant. The emulsifier in a cream formulation is generally a nonionic, anionic, cationic, or amphoteric surfactant.

As will be appreciated by those working in the field of pharmaceutical formulation, gels are semisolid, suspension-type systems. Single-phase gels contain organic macromolecules distributed substantially uniformly throughout the carrier liquid, which is typically aqueous, but also, preferably, contain an alcohol and, optionally, an oil. Preferred “organic macromolecules,” i.e., gelling agents, are crosslinked acrylic acid polymers such as the “carbomer” family of polymers, e.g., carboxypolyalkylenes that may be obtained commercially under the Carbopol® trademark. Also preferred are hydrophilic polymers such as polyethylene oxides, polyoxyethylene-polyoxypropylene copolymers, and polyvinylalcohol; cellulosic polymers such as hydroxypropyl cellulose, hydroxyethyl cellulose, hydroxypropyl methylcellulose, hydroxypropyl methylcellulose phthalate, and methyl cellulose; gums such as tragacanth and xanthan gum; sodium alginate; and gelatin. In order to prepare a uniform gel, dispersing agents such as alcohol or glycerin can be added, or the gelling agent can be dispersed by trituration, mechanical mixing or stirring, or combinations thereof.

Lotions, which are preferred for delivery of cosmetic agents, are preparations to be applied to the skin surface without friction, and are typically liquid or semiliquid preparations in which solid particles, including the active agent, are present in a water or alcohol base. Lotions are usually suspensions of solids, and preferably, for the present purpose, comprise a liquid oily emulsion of the oil-in-water type. Lotions are preferred formulations herein for treating large body areas, because of the ease of applying a more fluid composition. It is generally necessary that the insoluble matter in a lotion be finely divided. Lotions will typically contain suspending agents to produce better dispersions as well as compounds useful for localizing and holding the active agent in contact with the skin, e.g., methylcellulose, sodium carboxymethyl-cellulose, or the like.

Pastes are semisolid dosage forms in which the active agent is suspended in a suitable base. Depending on the nature of the base, pastes are divided between fatty pastes or those made from a single-phase aqueous gels. The base in a fatty paste is generally petrolatum or hydrophilic petrolatum or the like. The pastes made from single-phase aqueous gels generally incorporate carboxymethylcellulose or the like as a base.

Plasters are comprised of a pasty mixture that is spread on the body, either directly or after being saturated into a base material such as cloth. Medications, including the pharmacologically active bases of the invention, may be dissolved or dispersed within the plaster to make a medicated plaster.

Bioadhesives are preparations that adhere to surfaces of body tissues. Polymeric bioadhesive formulations are well known in the art; see, for example, Heller et al., “Biodegradable polymers as drug delivery systems”, in Chasin, M. and Langer, R., eds.: Dekker, N.Y., pp. 121-161 (1990); and U.S. Pat. No. 6,201,065. Suitable non-polymeric bioadhesives are also known in the art, including certain fatty acid esters (U.S. Pat. No. 6,228,383).

Formulations may also be prepared with liposomes, micelles, and microspheres. Liposomes are microscopic vesicles having a lipid wall comprising a lipid bilayer, and can be used as drug delivery systems herein as well. Generally, liposome formulations are preferred for poorly soluble or insoluble pharmaceutical agents. Liposomal preparations for use in the instant invention include cationic (positively charged), anionic (negatively charged) and neutral preparations. Cationic liposomes are readily available. For example, N[1-2,3-dioleyloxy)propyl]-N,N,N-triethylammonium (DOTMA) liposomes are available under the tradename Lipofectin® (GIBCO BRL, Grand Island, N.Y.). Similarly, anionic and neutral liposomes are readily available as well, e.g., from Avanti Polar Lipids (Birmingham, Ala.), or can be easily prepared using readily available materials. Such materials include phosphatidyl choline, cholesterol, phosphatidyl ethanolamine, dioleoylphosphatidyl choline (DOPC), dioleoylphosphatidyl glycerol (DOPG), dioleoylphoshatidyl ethanolamine (DOPE), among others. These materials can also be mixed with DOTMA in appropriate ratios. Methods for making liposomes using these materials are well known in the art.

Micelles are known in the art to be comprised of surfactant molecules arranged so that their polar headgroups form an outer spherical shell, while the hydrophobic, hydrocarbon chains are oriented towards the center of the sphere, forming a core. Micelles form in an aqueous solution containing surfactant at a high enough concentration so that micelles naturally result. Surfactants useful for forming micelles include, but are not limited to, potassium laurate, sodium octane sulfonate, sodium decane sulfonate, sodium dodecane sulfonate, sodium lauryl sulfate, docusate sodium, decyltrimethylammonium bromide, dodecyltrimethylammonium bromide, tetradecyltrimethylammonium bromide, tetradecyltrimethylammonium chloride, dodecylammonium chloride, polyoxyl 8 dodecyl ether, polyoxyl 12 dodecyl ether, nonoxynol 10 and nonoxynol 30. Micelle formulations can be used in conjunction with the present invention either by incorporation into the reservoir of a topical or transdermal delivery system, or into a formulation to be applied to the body surface.

Microspheres, similarly, may be incorporated into the present formulations and drug delivery systems. Like liposomes and micelles, microspheres essentially encapsulate a drug or drug-containing formulation. Microspheres are generally, although not necessarily, formed from synthetic or naturally occurring biocompatible polymers, but may also be comprised of charged lipids such as phospholipids. Preparation of microspheres is well known in the art and described in the pertinent texts and literature.

Various additives, known to those skilled in the art, may be included in the topical formulations. For example, solvents, including relatively small amounts of alcohol, may be used to solubilize certain formulation components. Although the permeation-enhancing bases herein are quite effective in enhancing drug penetration into the skin, it may be desirable, particularly with weaker bases or areas of thick or highly keratinized skin, to include an added permeation enhancer in the formulation. Examples of suitable enhancers include, but are not limited to, ethers such as diethylene glycol monoethyl ether (available commercially as Transcutol®) and diethylene glycol monomethyl ether; surfactants such as sodium laurate, sodium lauryl sulfate, cetyltrimethylammonium bromide, benzalkonium chloride, Poloxamer (231, 182, 184), Tween (20, 40, 60, 80), and lecithin (U.S. Pat. No. 4,783,450); alcohols such as ethanol, propanol, octanol, benzyl alcohol, and the like; polyethylene glycol and esters thereof such as polyethylene glycol monolaurate (PEGML; see, e.g., U.S. Pat. No. 4,568,343); amides and other nitrogenous compounds such as urea, dimethylacetamide (DMA), dimethylformamide (DMF), 2-pyrrolidone, 1-methyl-2-pyrrolidone, ethanolamine, diethanolamine and triethanolamine; terpenes; alkanones; and organic acids, particularly citric acid and succinic acid. Azone® and sulfoxides such as DMSO and C₁₀MSO may also be used, but are less preferred.

Most preferred enhancers are those lipophilic co-enhancers typically referred to as “plasticizing” enhancers, i.e., enhancers that have a molecular weight in the range of about 150 to 1000, an aqueous solubility of less than about 1 wt. %, preferably less than about 0.5 wt. %, and most preferably less than about 0.2 wt. %. The Hildebrand solubility parameter of plasticizing enhancers is in the range of about 2.5 to about 10, preferably in the range of about 5 to about 10. Such enhancers are described in detail in commonly assigned U.S. patent application Ser. No. 09/738,410, filed on Dec. 14, 2000, and in International Patent Application No. PCT/US00/34483, published Jun. 21, 2001 as WO 01/43775 A2. Preferred lipophilic enhancers are fatty esters, fatty alcohols, and fatty ethers. Examples of specific and most preferred fatty acid esters include methyl laurate, ethyl oleate, propylene glycol monolaurate, propylene glycerol dilaurate, glycerol monolaurate, glycerol monooleate, isopropyl n-decanoate, and octyldodecyl myristate. Fatty alcohols include, for example, stearyl alcohol and oleyl alcohol, while fatty ethers include compounds wherein a diol or triol, preferably a C₂-C₄ alkane diol or triol, are substituted with one or two fatty ether substituents.

Additional permeation enhancers will be known to those of ordinary skill in the art of topical drug delivery, and/or are described in the pertinent texts and literature. See, e.g., Percutaneous Penetration Enhancers, Smith et al., eds. (CRC Press, 1995).

The present formulations may also include conventional additives such as opacifiers, antioxidants, fragrance, colorants, gelling agents, thickening agents, stabilizers, surfactants, and the like. Other agents may also be added, such as antimicrobial agents, to prevent spoilage upon storage, i.e., to inhibit growth of microbes such as yeasts and molds. Suitable antimicrobial agents are typically selected from the group consisting of the methyl and propyl esters of p-hydroxybenzoic acid (i.e., methyl and propyl paraben), sodium benzoate, sorbic acid, imidurea, and combinations thereof.

The formulations may also contain irritation-mitigating additives to minimize or eliminate the possibility of skin irritation or skin damage resulting from the permeation-enhancing base or other components of the composition. Suitable irritation-mitigating additives include, for example: -tocopherol; monoamine oxidase inhibitors, particularly phenyl alcohols such as 2-phenyl-1-ethanol; glycerin; salicylic acids and salicylates; ascorbic acids and ascorbates; ionophores such as monensin; amphiphilic amines; ammonium chloride; N-acetylcysteine; cis-urocanic acid; capsaicin; and chloroquine. The irritant-mitigating additive, if present, may be incorporated into the present formulations at a concentration effective to mitigate irritation or skin damage, typically representing not more than about 20 wt. %, more typically not more than about 5 wt. %, of the composition.

The composition of the invention may also be administered through the skin or mucosal tissue using a conventional skin patch, wherein the composition is contained within a laminated structure that serves as a drug delivery device to be affixed to the body surface. In such a structure, the pharmaceutical formulation is contained in a layer, or “reservoir,” underlying an upper backing layer. The laminated structure may contain a single reservoir, or it may contain multiple reservoirs.

In one embodiment, the reservoir comprises a polymeric matrix of a pharmaceutically acceptable adhesive material that serves to affix the system to the skin during drug delivery; typically, the adhesive material is a pressure-sensitive adhesive (PSA) that is suitable for long-term skin contact, and that should be physically and chemically compatible with the pharmacologically active base and any carriers, vehicles or-other additives that are present. Examples of suitable adhesive materials include, but are not limited to, the following: polyethylenes; polysiloxanes; polyisobutylenes; polyacrylates; polyacrylamides; polyurethanes; plasticized ethylene-vinyl acetate copolymers; and tacky rubbers such as polyisobutene, polybutadiene, polystyrene-isoprene copolymers, polystyrene-butadiene copolymers, and neoprene (polychloroprene). Preferred adhesives are polyisobutylenes.

The backing layer functions as the primary structural element of the transdermal system and provides the device with flexibility and, preferably, occlusivity. The material used for the backing layer should be inert and incapable of absorbing drug, base, or components of the formulation contained within the device. The backing is preferably comprised of a flexible elastomeric material that serves as a protective covering to prevent loss of drug and/or vehicle via transmission through the upper surface of the patch, and will preferably impart a degree of occlusivity to the system, such that the area of the body surface covered by the patch becomes hydrated during use. The material used for the backing layer should permit the device to follow the contours of the skin and wart and be worn comfortably on areas of skin such as at joints or other points of flexure, that are normally subjected to mechanical strain, with little or no likelihood of the device disengaging from the skin due to differences in the flexibility or resiliency of the skin and the device. The materials used as the backing layer are either occlusive or permeable, as noted above, although occlusive backings are preferred, and are generally derived from synthetic polymers (e.g., polyester, polyethylene, polypropylene, polyurethane, polyvinylidine chloride, and polyether amide), natural polymers (e.g., cellulosic materials), or macroporous woven and nonwoven materials.

During storage and prior to use, the laminated structure includes a release liner. Immediately prior to use, this layer is removed from the device so that the system may be affixed to the skin. The release liner should be made from a drug/vehicle impermeable material, and is a disposable element that serves only to protect the device prior to application. Typically, the release liner is formed from a material impermeable to the pharmacologically active agent and the base, and which is easily stripped from the patch prior to use.

In an alternative embodiment, the reservoir containing the active agent plus base and the skin contact adhesive are present as separate and distinct layers, with the adhesive underlying the reservoir. In such a case, the reservoir may be a polymeric matrix as described above. Alternatively, the reservoir may be comprised of a liquid or semisolid formulation contained in a closed compartment or “pouch,” or it may be a hydrogel reservoir, or it may take some other form. Hydrogel reservoirs are particularly preferred herein. As will be appreciated by those skilled in the art, hydrogels are macromolecular networks that absorb water and thus swell but do not dissolve in water. That is, hydrogels contain hydrophilic functional groups that provide for water absorption, but the hydrogels are comprised of crosslinked polymers that give rise to aqueous insolubility. Generally, then, hydrogels are comprised of crosslinked hydrophilic polymers such as a polyurethane, a polyvinyl alcohol, a polyacrylic acid, a polyoxyethylene, a polyvinylpyrrolidone, a poly(hydroxyethyl methacrylate) (poly(HEMA)), or a copolymer or mixture thereof. Particularly preferred hydrophilic polymers are copolymers of HEMA and polyvinylpyrrolidone.

Additional layers, e.g., intermediate fabric layers and/or rate-controlling membranes, may also be present in any of these drug delivery systems. Fabric layers may be used to facilitate fabrication of the device, while a rate-controlling membrane may be used to control the rate at which a component permeates out of the device. The component may be an active agent, an enhancer, or some other component contained in the drug delivery system. A rate-controlling membrane, if present, will be included in the system on the skin side of one or more of the drug reservoirs. The materials used to form such a membrane are selected to limit the flux of one or more components contained in the drug formulation. Representative materials useful for forming rate-controlling membranes include polyolefins such as polyethylene and polypropylene, polyamides, polyesters, ethylene-ethacrylate copolymer, ethylene-vinyl acetate copolymer, ethylene-vinyl methylacetate copolymer, ethylene-vinyl ethylacetate copolymer, ethylene-vinyl propylacetate copolymer, polyisoprene, polyacrylonitrile, ethylene-propylene copolymer, and the like.

Generally, the underlying surface of the transdermal device, i.e., the skin contact area, has an area in the range of about 0.25 cm² to 200 cm², preferably 1 cm² to 25 cm², more preferably 2 cm² to 10 cm². That area will vary, of course, with the size of the area to be treated. Larger patches will be necessary to accommodate larger areas of affected skin, whereas smaller patches can be used for smaller areas of affected skin.

Such drug delivery systems may be fabricated using conventional coating and laminating techniques known in the art. For example, adhesive matrix systems can be prepared by casting a fluid admixture of adhesive, active agent and vehicle onto the backing layer, followed by lamination of the release liner. Similarly, the adhesive mixture may be cast onto the release liner, followed by lamination of the backing layer. Alternatively, the drug reservoir may be prepared in the absence of drug or excipient, and then loaded by “soaking” in a drug/vehicle mixture. In general, these patches are fabricated by solvent evaporation, film casting, melt extrusion, thin film lamination, die cutting, or the like. The active agent will generally be incorporated into the device during patch manufacture rather than subsequent to preparation of the device.

In a preferred delivery system, an adhesive overlayer that also serves as a backing for the delivery system is used to better secure the patch to the body surface. This overlayer is sized such that it extends beyond the drug reservoir so that adhesive on the overlayer comes into contact with the body surface. The overlayer is useful because the adhesive/drug reservoir layer may lose its adhesion a few hours after application due to hydration. By incorporating such adhesive overlayer, the delivery system remains in place for the required period of time.

Other types and configurations of topically applied drug delivery systems may also be used in conjunction with the present invention, as will be appreciated by those skilled in the art of topical drug delivery. See, for example, Ghosh, Transdermal and Topical Drug Delivery Systems (Interpharm Press, 1997), particularly Chapters 2 and 8.

V. Administration:

The method of delivery of the pharmaceutically active composition may vary, but necessarily involves application of a formulation of the invention to an area of body surface affected with an inflammatory dermatosis. A cream, ointment, paste, plaster, or lotion may be spread on the affected area of skin and gently rubbed in. Similarly, a polymeric or other bioadhesive formulation may be spread or dabbed on the affected area of skin. A solution may be applied in the same ways, but more typically will be applied with a dropper, swab, or the like, and carefully applied to the affected area of skin. Petrolatum may be spread on the skin surrounding the affected area of skin to protect it from possible irritation during treatment.

The dosing regimen will depend on a number of factors that may readily be determined, such as the size of the affected area, the severity of the dermatosis, and the responsiveness of the inflammatory dermatosis to treatment, but will normally be one or more doses per day, with a course of treatment lasting from several days to several months, or until a cure is effected or a significant diminution in the size and/or severity of the inflammatory dermatosis is achieved. One of ordinary skill may readily determine optimum dosages, dosing methodologies, and repetition rates. In general, it is contemplated that the formulation will be applied one to four times daily. With a skin patch, which is a preferred embodiment, the device is generally maintained in place on the body surface throughout a drug delivery period, typically in the range of 8 to 72 hours, and replaced as necessary.

It is to be understood that while the invention has been described in conjunction with the preferred specific embodiments thereof, the foregoing description is intended to illustrate and not limit the scope of the invention. Other aspects, advantages, and modifications will be apparent to those skilled in the art to which the invention pertains. Furthermore, the practice of the present invention will employ, unless otherwise indicated, conventional techniques of drug formulation, particularly topical and transdermal drug formulation, which are within the skill of the art. Such techniques are fully explained in the literature. See Remington: The Science and Practice of Pharmacy, cited supra, as well as Goodman & Gilman's The Pharmacological Basis of Therapeutics, 9th Ed. (New York: McGraw-Hill, 1996).

All patents, patent applications, and publications mentioned herein are hereby incorporated by reference in their entireties.

### Experimental

The practice of the present invention will employ, unless otherwise indicated, conventional techniques of pharmaceutical formulation and the like, which are within the skill of the art. Such techniques are fully explained in the literature. In the following examples, efforts have been made to ensure accuracy with respect to numbers used (e.g., amounts, temperatures, etc.) but some experimental error and deviation should be accounted for. Unless otherwise indicated, temperature is in degrees Celsius and pressure is at or near atmospheric pressure at sea level. All reagents were obtained commercially unless otherwise indicated.

### EXAMPLE 1

A topical gel of the invention is prepared by conventional pharmaceutical methods. The indicated amounts of the following ingredients are used:

| Ingredient | Amount | |
|---|---|---|
| Purified water | 600 | grams |
| Polyethylene glycol | 400 | grams |
| Potassium hydroxide | 0.01 | gram |
| Adapalene | 1.0 | gram |
| Edetate disodium | 0.1 | gram |
| Carbomer 934P | 12.5 | grams |
| Poloxamer 407 | 2.0 | grams |
| Polysorbate 40 | 2.0 | grams |
| Butylated hydroxytoluene | 0.5 | grams |
| Benzyl alcohol | 10.0 | grams |

The carbomer 934P and the edetate disodium are added to 225 mL of the purified water, and the mixture is homogenized at low speed until the carbomer is dispersed. Next, the polaxamer 407, mixed with 225 mL of the purified water, is added to the carbomer mixture, and the resulting mixture is homogenized at low speed. The adapalene, suspended in 50 mL of the purified water, is then added to this mixture, which is homogenized at low speed. The potassium hydroxide, dissolved in 100 mL of purified water, is further added to this mixture, and the resulting mixture (Mixture 1) is homogenized at low speed. In a separate container, the polysorbate 40 and the butylated hydroxytoluene are added to the polyethylene glycol, and the resulting mixture is heated to 65° C. and maintained at this temperature until all the compounds are dissolved; this mixture is then allowed to cool to room temperature, at which time the benzyl alcohol is added, and the resulting mixture is homogenized at low speed. This mixture is then added to Mixture 1, and the resulting mixture is mixed at low speed until it is homogeneous, forming a gel of the invention.

### EXAMPLE 2

A topical cream of the invention is prepared by conventional pharmaceutical methods. The indicated amounts of the following ingredients are used:

| Ingredient | Amount | |
|---|---|---|
| Purified water | 370 | grams |
| White petrolatum | 250 | grams |
| Stearyl alcohol | 250 | grams |
| Propylene glycol | 120 | grams |
| Sodium lauryl sulfate | 10 | grams |
| Adapalene | 1 | grams |
| Methylparaben | 0.25 | gram |
| Propylparaben | 0.15 | gram |
| Potassium hydroxide | 0.01 | gram |

The stearyl alcohol and the white petrolatum are melted together on a steam bath, and then maintained at a temperature of approximately 75° C. The other ingredients are then added, after previously having been dissolved or suspended in the purified water and warmed to 75° C., and the resulting mixture is stirred until it congeals into a cream of the invention.

### EXAMPLE 3

A skin patch of the invention may be prepared by conventional pharmaceutical methods. A square piece of sterile, finely woven gauze one centimeter on each side is placed in the center of a square piece of occlusive surgical adhesive tape two centimeters on each side. To the gauze is applied 0.4 mL of the gel of Example 1; the gel is allowed to soak into the gauze. This skin patch of the invention is used within three hours of preparation.

### EXAMPLE 4

The gel formulation of Example 1 is provided to patients having facial acne vulgaris. An affected area of skin on each patient is selected for treatment, and this area is photographed before treatment begins. Each of the patients is instructed to topically apply the formulation of the invention to the affected area twice daily for eight weeks. The patients return to the clinic every seven days, when the affected area is again photographed. It is found that most of the acne lesions are significantly reduced in size and redness after two weeks and that the treated areas of skin are essentially free of acne lesions after eight weeks of treatment, with no scarring, pigmentation changes, or other undesirable side effects.

### EXAMPLE 5

The skin patches of Example 3 are provided to patients having facial acne vulgaris. An affected area of skin on each patient is selected for treatment, and this area is photographed before treatment begins. Each of the patients is instructed to topically apply a skin patch of the invention over the affected area, replacing the old patch with a new one every 48 hours. The patients return to the clinic every seven days, when the affected area is again photographed. It is found that most of the acne lesions are significantly reduced in size and redness after two weeks and that the treated areas of skin are essentially free of acne lesions after eight weeks of treatment, with no scarring, pigmentation changes, or other undesirable side effects.

### EXAMPLE 6

The gel formulation of Example 1 is provided in ajar labeled “A” to about eight patients, each of whom has acne vulgaris on both sides of his or her face. An otherwise identical gel lacking the permeation-enhancing base and active agent acts as a placebo and is also provided to the patients, in ajar labeled “B”. Neither the patients nor those administering the jars to the patients know which jars contain the placebo and which contain the base and active agent; such records are kept hidden from these individuals throughout the duration of the trial. An affected area of skin on each side of the face of each patient is selected for treatment, and these areas are photographed before treatment begins. Each participating patient is instructed to topically apply the gel of jar “A” to the affected area of the right side of the face and the gel of jar “B” to the affected area of the left side of the face twice daily for eight weeks. The patients return to the clinic every seven days, when the affected areas are again measured and photographed. It is found that most of the acne lesions treated with the gel of the invention are significantly reduced in size and redness after two weeks and that the treated areas of skin are essentially free of acne lesions after eight weeks of treatment, with no scarring, pigmentation changes, or other undesirable side effects, whereas the affected areas treated with placebo continue to have acne lesions throughout the course of the study.

We claim:

1. A method of treating an individual afflicted with an inflammatory dermatosis, comprising topically administering to a localized region affected by the inflammatory dermatosis on the individual's body surface a formulation comprised of an active agent effective in treating the inflammatory dermatosis, a pharmaceutically acceptable topical carrier, and a permeation-enhancing base, the base being present in a predetermined amount effective to enhance the flux of the active agent through the localized region of the body surface without causing damage thereto.

2. The method of claim 1, wherein the predetermined amount of the permeation-enhancing base is effective to provide a pH in the range of approximately 8.0 to 13 at the localized region of the body surface, during drug administration.

3. The method of claim 2, wherein the pH is in the range of approximately 8.0 to 11.5.

4. The method of claim 2, wherein the pH is in the range of approximately 8.5 to 10.5.

5. The method of claim 1, wherein the inflammatory dermatosis is selected from the group consisting of allergic dermatoses, pruritic dermatoses, vascular dermatoses, sebaceous gland disorders, papulosquamous dermatoses, bacterial dermatoses, viral dermatoses, mycolic skin infections, granulomatous dermatoses, parasitic skin dermatoses, exfoliative dermatitis, bullous dermatoses, pigmented dermatoses, photosensitive dermatoses, dermatoses caused by collagen diseases, and dermatoses due to internal diseases.

6. The method of claim 5, wherein the inflammatory dermatosis is a sebaceous gland disorder.

7. The method of claim 6, wherein the sebaceous gland disorder is an acneiform disorder.

8. The method of claim 4, wherein the acneiform disorder is selected from the group consisting of acne vulgaris, acne conglombata, hidradenitis suppurativa, acne rosacea, seborrhea, seborrheic dermatitis, gram negative folliculitis, pyoderma faciale, steatocystoma multiplex, sebaceous hyperplasia, and rhinophyma.

9. The method of claim 8, wherein the acneiform disorder is acne vulgaris.

10. The method of claim 5, wherein the inflammatory dermatosis is a papulosquamous dermatosis.

11. The method of claim 10, wherein the papulosquamous dermatosis is psoriasis.

12. The method of claim 1, wherein the inflammatory dermatosis is an autoimmune condition.

13. The method of claim 12, wherein the autoimmune condition is atopic dermatitis, mast cell disease, bullous pemphigoid, pemphigus vulgaris, necrotizing vasculitis, discoid lupus erythematosus, systemic lupus erythematosis, or dermatitis herpetiformis.

14. The method of claim 1, wherein the formulation is aqueous.

15. The method of claim 14, wherein the aqueous formulation is selected from the group consisting of a cream, a gel, a lotion, a paste, and a solution.

16. The method of claim 14, wherein the aqueous formulation is a cream.

17. The method of claim 14, wherein the aqueous formulation is a gel.

18. The method of claim 14, wherein the aqueous formulation is a lotion.

19. The method of claim 14, wherein the aqueous formulation is a solution.

20. The method of claim 14, wherein the aqueous formulation is a paste.

21. The method of claim 1, wherein the formulation is a bioadhesive.

22. The method of claim 1, wherein the formulation is in a medicated plaster.

23. The method of claim 1, wherein the formulation is in a skin patch.

24. The method of claim 1, wherein the permeation-enhancing base is a base.

25. The method of claim 24, wherein the base is selected from the group consisting of inorganic hydroxides, inorganic oxides, metal salts of weak acids, and mixtures thereof.

26. The method of claim 25, wherein the base is an inorganic hydroxide.

27. The method of claim 26, wherein the inorganic hydroxide is selected from the group consisting of ammonium hydroxide, alkali metal hydroxides, alkaline earth metal hydroxides, and mixtures thereof.

28. The method of claim 27, wherein the inorganic hydroxide is selected from the group consisting of ammonium hydroxide, sodium hydroxide, calcium hydroxide, potassium hydroxide, magnesium hydroxide, and mixtures thereof.

29. The method of claim 28, wherein the inorganic hydroxide is sodium hydroxide.

30. The method of claim 24, wherein the base is an inorganic oxide.

31. The method of claim 24, wherein the base is a metal salt of a weak acid.

32. The method of claim 1, wherein the permeation-enhancing base is a nitrogenous base.

33. The method of claim 1, wherein the permeation-enhancing base is an organic base.

34. The method of claim 33, wherein the organic base is selected from the group consisting of primary amines, secondary amines, tertiary amines, amides, oximes, nitrogen-containing heterocycles, and urea.

35. The method of claim 34, wherein the organic base is a primary amine, a secondary amine, or a tertiary amine.

36. The method of claim 35, wherein the organic base has the structure NR¹R²R³, wherein R¹, R² and R³ are selected from H, alkyl, hydroxyalkyl, alkoxyalkyl, alkenyl, hydroxyalkenyl, alkoxyalkenyl, cycloalkyl, cycloalkyl-substituted alkyl, monocyclic aryl, and monocyclic aryl-substituted alkyl, with the proviso that at least one of R¹, R² and R³ is other than H.

37. The method of claim 35, wherein the organic base is selected from the group consisting of diethanolamine, triethanolamine, isopropanolamine, triisopropanolamine, dibutanol amine, tributanol amine, N-dodecylethanolamine, N-(2-methoxyethyl) dodecylamine, N-(2,2-dimethoxyethyl)dodecylamine, N-ethyl-N-(dodecyl)ethanolamine, N-ethyl-N-(2-methoxyethyl)dodecylamine, N-ethyl-N-(2,2-dimethoxyethyl) dodecylamine, dimethyldodecylamine-N-oxide, monolauroyl lysine, dipalmitoyl lysine, dodecylamine, stearylamine, phenylethylamine, triethylamine, PEG-2 oleamine, PEG-5 oleamine, dodecyl 2-(N,N-dimethylamino)propionate, bis(2-hydroxyethyl)oleylamine, and combinations thereof.

38. The method of claim 34, wherein the organic base is an amide.

39. The method of claim 38, wherein the amide has the structure R⁴—(CO)—NR⁵R⁶ where R⁴, R⁵ and R⁶ are independently selected from H, alkyl, cycloalkyl, cycloalkyl-substituted alkyl, monocyclic aryl, and monocyclic aryl-substituted alkyl.

40. The method of claim 39, wherein the amide is selected from the group consisting of hexamethyleneacetamide, hexamethyleneoctamide, hexamethylene lauramide, hexamethylene palmitamide, N,N-dimethyl formamide, N,N-dimethyl acetamide, N,N-dimethyloctamide, N,N-dimethyidecamide, toluamide, dimethyl-m-toluamide, diethyl-m-toluamide, and combinations thereof.

41. The method of claim 34, wherein the organic base is a nitrogen-containing heterocycle.

42. The method of claim 41, wherein the nitrogen-containing heterocycle is selected from the group consisting of 2-pyrrolidone, 1-methyl-2-pyrrolidone, 5-methyl-2-pyrrolidone, 1,5-dimethyl-2-pyrrolidone, 1-ethyl-2-pyrrolidone, 1-propyl-3-dodecylpyrrolidine, 1-dodecyclazacycloheptan-2-one, ethylene thiourea, hydantoin, oxalylurea, imidazolidilyl urea, N-octadecyl morpholine, dodecylpyridinium, N-dodecylpyrrolidine, N-dodecylpiperidine, N-dodecylhomopiperidine, and combinations thereof.

43. The method of claim 1, wherein the active agent is an antibacterial agent.

44. The method of claim 43, wherein the antibacterial agent is selected from the group consisting of erythromycin, azelaic acid, clindamycin, tetracycline, minocycline, nadifloxacin, cephalexin, doxycycline, oflaxacin, and sulfonamides, or a combination thereof.

45. The method of claim 44, wherein the antibacterial agent is erythromycin.

46. The method of claim 44, wherein the antibacterial agent is clindamycin.

47. The method of claim 44, wherein the antibacterial agent is a sulfonamide.

48. The method of claim 47, wherein the sulfonamide is sodium sulfacetamide.

49. The method of claim 1, wherein the active agent is benzoyl peroxide.

50. The method of claim 1, wherein the active agent is sulfur.

51. The method of claim 1, wherein the active agent is salicylic acid.

52. The method of claim 1, wherein the active agent is a retinoid.

53. The method of claim 52, wherein the retinoid is selected from the group consisting of tretinoin, adapalene, and tazarotene, or a combination thereof.

54. The method of claim 53, wherein the retinoid is tretinoin.

55. The method of claim 53, wherein the retinoid is adapalene.

56. The method of claim 1, wherein the active agent is resorcinol.

57. The method of claim 1, wherein the active agent is a corticosteroid.

58. The method of claim 57, wherein the corticosteroid is triamcinolone.

59. The method of claim 1, wherein the active agent is an alpha hydroxy acid.

60. The method of claim 1, wherein the active agent is an alpha keto acid.

61. The method of claim 1, wherein the formulation includes one or more additional active agents effective in treating inflammatory dermatoses.

62. The method of claim 1, wherein the formulation is applied periodically over an extended time period.

63. The method of claim 1, wherein the formulation is applied approximately twice weekly.

64. The method of claim 1, wherein the formulation is applied once daily.

65. The method of claim 1, wherein the formulation is applied twice daily.

66. The method of claim 1, wherein the formulation is applied on an as-needed basis.

67. The method of claim 62, wherein said extended time period is at least three months.

68. The method of claim 67, wherein said extended time period is at least four months.

69. The method of claim 1, wherein the formulation is administered by applying a drug delivery device to the localized region of the patient's body surface thereby forming a body surface-delivery device, the device comprising the formulation, and having an outer backing layer that serves as the outer surface of the device during use.

70. A composition of matter useful for the topical treatment of an inflammatory dermatosis comprising a formulation of:
(a) a therapeutically effective amount of an active agent effective in treating an inflammatory dermatosis;
(b) a permeation-enhancing base in an amount effective to enhance the flux of the active agent through the body surface without causing damage thereto; and
(c) a pharmaceutically acceptable carrier suitable for topical drug administration.

71. The composition of claim 70, wherein the pH is in the range of approximately 8.0 to 13.

72. The composition of claim 71, wherein the pH is in the range of approximately 8.0 to 11.5.

73. The composition of claim 72, wherein the pH is in the range of approximately 8.5 to 10.5.

74. The composition of claim 70, wherein the carrier is aqueous.

75. The composition of claim 74, selected from the group consisting of a cream, a gel, a lotion, and a paste.

76. The composition of claim 75, in the form of a cream.

77. The composition of claim 75, in the form of a gel.

78. The composition of claim 75, in the form of a lotion.

79. The composition of claim 75, in the form of a paste.

80. The composition of claim 70, in the form of a bioadhesive.

81. The composition of claim 70, in a medicated plaster.

82. The composition of claim 70, in a skin patch.

83. The composition of claim 70, wherein the permeation-enhancing base is a base.

84. The composition of claim 83, wherein the base is selected from the group consisting of inorganic hydroxides, inorganic oxides, metal salts of weak acids, and mixtures thereof.

85. The composition of claim 84, wherein the base is an inorganic hydroxide.

86. The composition of claim 85, wherein the inorganic hydroxide is selected from the group consisting of ammonium hydroxide, alkali metal hydroxides, alkaline earth metal hydroxides, and mixtures thereof.

87. The composition of claim 85, wherein the inorganic hydroxide is selected from the group consisting of ammonium hydroxide, sodium hydroxide, calcium hydroxide, potassium hydroxide, magnesium hydroxide, and mixtures thereof.

88. The formulation of claim 87, wherein the inorganic hydroxide is sodium hydroxide.

89. The composition of claim 84, wherein the base is an inorganic oxide.

90. The composition of claim 84, wherein the base is a metal salt of a weak acid.

91. The composition of claim 70, wherein the permeation-enhancing base is a nitrogenous base.

92. The composition of claim 70, wherein the permeation-enhancing base is an organic base.

93. The composition of claim 92, wherein the organic base is selected from the group consisting of primary amines, secondary amines, tertiary amines, amides, oximes, nitrogen-containing heterocycles, and urea.

94. The composition of claim 93, wherein the organic base is a primary amine, a secondary amine,or a tertiary-amine.

95. The composition of claim 94, wherein the organic base has the structure NR¹R²R³ wherein R¹, R² and R³ are selected from H, alkyl, hydroxyalkyl, alkoxyalkyl, alkenyl, hydroxyalkenyl, alkoxyalkenyl, cycloalkyl, cycloalkyl-substituted alkyl, monocyclic aryl, and monocyclic aryl-substituted alkyl, with the proviso that at least one of R¹, R² and R³ is other than H.

96. The composition of claim 94, wherein the organic base is selected from the group consisting of diethanolamine, triethanolamine, isopropanolamine, triisopropanolamine, dibutanol amine, tributanol amine, N-dodecylethanolamine, N-(2-methoxyethyl) dodecylamine, N-(2,2-dimethoxyethyl)dodecylamine, N-ethyl-N-(dodecyl)ethanolamine, N-ethyl-N-(2-methoxyethyl)dodecylamine, N-ethyl-N-(2,2-dimethoxyethyl) dodecylamine, dimethyldodecylamine-N-oxide, monolauroyl lysine, dipalmitoyl lysine, dodecylamine, stearylamine, phenylethylamine, triethylamine, PEG-2 oleamine, PEG-5 oleamine, dodecyl 2-(N,N-dimethylamino)propionate, bis(2-hydroxyethyl)oleylamine, and combinations thereof.

97. The composition of claim 93, wherein the organic base is an amide.

98. The composition of claim 97, wherein the amide has the structure R⁴—(CO)—NR⁵R⁶ where R⁴, R⁵ and R⁶ are independently selected from H, alkyl, cycloalkyl, cycloalkyl-substituted alkyl, monocyclic aryl, and monocyclic aryl-substituted alkyl.

99. The composition of claim 98, wherein the amide is selected from the group consisting of hexamethyleneacetamide, hexamethyleneoctamide, hexamethylene lauramide, hexamethylene palmitamide, N,N-dimethyl formamide, N,N-dimethyl acetamide, N,N-dimethyloctamide, N,N-dimethyldecamide, toluamide, dimethyl-m-toluamide, diethyl-m-toluamide, and combinations thereof.

100. The composition of claim 93, wherein the organic base is a nitrogen-containing heterocycle.

101. The composition of claim 100, wherein the nitrogen-containing heterocycle is selected from the group consisting of 2-pyrrolidone, 1-methyl-2-pyrrolidone, 5-methyl-2-pyrrolidone, 1,5-dimethyl-2-pyrrolidone, 1-ethyl-2-pyrrolidone, 1-propyl-3-dodecylpyrrolidine, 1-dodecyclazacycloheptan-2one, ethylene thiourea, hydantoin, oxalylurea, imidazolidilyl urea, N-octadecyl morpholine, dodecylpyridinium, N-dodecylpyrrolidine, N-dodecylpiperidine, N-dodecylhomopiperidine, and combinations thereof.

102. The composition of claim 70, wherein the active agent is an antibacterial agent.

103. The composition of claim 102, wherein the antibacterial agent is selected from the group consisting of erythromycin, azelaic acid, clindamycin, tetracycline, minocycline, nadifloxacin, cephalexin, doxycycline, oflaxacin, and sulfonamides, or a combination thereof.

104. The composition of claim 103, wherein the antibacterial agent is erythromycin.

105. The composition of claim 103, wherein the antibacterial agent is clindamycin.

106. The composition of claim 103, wherein the antibacterial agent is a sulfonamide.

107. The composition of claim 106, wherein the sulfonamide is sodium sulfacetamide.

108. The composition of claim 70, wherein the active agent is benzoyl peroxide.

109. The composition of claim 70, wherein the active agent is sulfur.

110. The composition of claim 70, wherein the active agent is salicylic acid.

111. The composition of claim 70, wherein the active agent is a retinoid.

112. The composition of claim 111, wherein the retinoid is selected from the group consisting of tretinoin, adapalene, and tazarotene, or a combination thereof.

113. The composition of claim 112, wherein the retinoid is tretinoin.

114. The composition of claim 112, wherein the retinoid is adapalene.

115. The composition of claim 70, wherein the active agent is resorcinol.

116. The composition of claim 70, wherein the active agent is a corticosteroid.

117. The composition of claim 116, wherein the corticosteroid is triamcinolone.

118. The composition of claim 70, wherein the active agent is an alpha hydroxy acid.

119. The composition of claim 70, wherein the active agent is an alpha keto acid.

120. The composition of claim 70, wherein the formulation includes one or more additional active agents effective in treating an inflammatory dermatosis.

121. The composition of claim 70, wherein the active agent is contained within liposomes, micelles, or microspheres.
